(19) United States
(12) Patent Application Publication
(10) Pub. No.: US 2021/0205807 A1
(43) Pub. Date: Jul. 8, 2021

(54) Acoustofluidic Platform for Fully Automated End-to-End Biological Sample Processing

(71) Applicant: Anubhav Dubey, Alpharetta, GA (US)

(72) Inventor: Anubhav Dubey, Alpharetta, GA (US)

(21) Appl. No.: 17/140,120

(22) Filed: Jan. 3, 2021

Related U.S. Application Data
(60) Provisional application No. 62/956,640, filed on Jan. 3, 2020.

(51) Int. Cl. B01L 3/00

(52) U.S. Cl. CPC B01L 3/502715; B01L 2200/028; B01L 2200/027

(57) ABSTRACT

The present invention relates to the use of microfluidics to introduce a sample and release a smaller subset of target molecule. In one embodiment of the present invention, a method of using microfluidics in order to separate and extract smaller subsets of target molecules from larger sample is described. In another embodiment of the present invention, a method of using microfluidic devices integrated with acoustics in order to fraction blood to serum to microRNA is described. In the aforementioned embodiment of the present invention, a method of using such devices for early stage diagnostics for an assortment of diseases is described.

### FIELD OF THE INVENTION

The present invention relates to devices, and more particularly, the creation and utilization of automatic microfluidic sample processing systems.

### BACKGROUND

Historically, sample processing has been overwhelmed with manual steps and the use of multiple kits. Long and tedious procedures are required for sample processing regardless of the input sample and the targeted output sample. Besides the challenges present when working with a single sample, there are various issues with reading and making sense of data from different kits, procedures, and platforms. As such, variability in sample processing and data collection across the research, clinical, and commercial space, has increased the need for automation. Automation in sample processing allows for quality control across samples from single-center studies and multi-center studies while maintaining the integrity of the procedure and data.

Some groups have developed fully automated platforms, but they have been largely based off of robotics. The challenge with robotic-based systems is that their physical size and cost create a large barrier for most research, clinical, or commercial settings. Moreover, its accuracy and precision have limitations especially given the introduction of inaccuracies and unwanted variables due to the moving parts. Although other platforms have been developed to further automate the process without robotics, they require external equipment and basic manual steps. Granted these systems provide a better solution in regard to accessibility, they continue to create variables and differences that directly impact the integrity of the data. As such, novel technologies are being developed, especially with the use and integration of microfluidics as it has shown great promise given its capabilities.

Microfluidics is the science of working with minute volumes of liquids using intricate and detailed channels that can quickly and cost-effectively be manufactured. Their ability to be simple and complex at the same time makes them applicable to a range of areas from basic science to processing systems to electronics. Microfluidics is especially relevant when it comes to automating procedures and processes that may contain a number of manual steps. Therefore, their application in biological sciences, moreover, sample processing can be valuable and advantageous.

Microfluidics in itself can be quite valuable, however, its integration with other techniques, approaches, and forces can make it more effective. They can be integrated with magnetic, thermal, acoustic chemical, electric, pressure, and/or mechanical systems and alongside techniques such as capillary forces, the Zweifach-Fung effect, dielectrophoresis, sedimentation, and membrane filtration. Exploiting both aspects with microfluidics allows for the creation of seamless, intricate, and encompassing devices.

The standard procedure for separating and extracting serum and then total RNA. from a sample of blood require numerous manual steps such as collection, centrifugation, separation, isolation kit, and extraction. Even if the final output is different and comes before the total RNA stage, sufficient manual work is involved to weaken the integrity and reproducibility of the data. As such, the use of microfluidics can successfully enhance the quality of the process and data. Furthermore, microfluidics increases the flexibility of the device in utilization and performance, and applicability in virtually all settings from the laboratory to the field. By using microfluidics, many fundamental issues in current sample processing techniques and make it more geared towards research, clinical, and commercial users.

### BRIEF SUMMARY OF THE INVENTION

The present invention. comprises a microfluidic device consisting of single or multiple stages with one inlet to introduce a sample and one output to release a subset of analytes. The stages comprise a channel and an integrated system. The stage's integrated system comprises acoustic, magnetic, thermal, chemical, electric, pressure, and/or mechanical systems. The stages are modules defined for different target subsets comprising serum, plasma, exosomes, polynucleotides, and/or polypeptides. The stages are connected and continuous. The analytes comprise blood fractions, polynucleotides, and/or polypeptides. The sample comprises blood, plasma, serum, urine, saliva, and tears. Other features and advantages of the invention will become apparent from the drawings and the detailed description.

### BRIEF DESCRIPTION OF THE DRAWINGS:

Embodiments are illustrated in the referenced figures. It is intended that the embodiments and figures disclosed herein are to be considered illustrative instead of restrictive.

FIG. 1 depicts, in accordance with an embodiment described herein, an illustration of the top view of a microfluidics device. (a) depicts an acoustic transducer used for the purpose of separating serum from a whole blood sample; (b) depicts the inlet through which samples will be introduced, and the beginning of the first module; (c) depicts a junction where the straight path leads to a collection deposit for heavier and irrelevant parts of the sample while the two bifurcations flow the serum portion of the sample; (d) depicts a junction where the serum sample from the two bifurcations meet and follow to a straight channel, and the end of the first module and the beginning of the second module; (e) depicts two sets of acoustic transducers on either side of a straight channel used for the purpose of releasing microRNAs from proteins and complexes in the serum channel; (f) depicts a filter that is used to separate relevant taiga molecules such as microRNAs from larger molecules such as proteins in the serum sample, and the end of the second module and the beginning of the third module; (g) depicts an intricate microfluidics channel laid over a microarray chip used for the purpose of hybridizing target microRNAs to capture probes; (h) depicts the slot through which chips are inserted and removed before and after hybridization, respectively, and the end of the third and final module.

FIG. 2 depicts, in accordance with an embodiment described herein, an illustration of the corner view of a microfluidics device. (a) depicts an acoustic transducer used for the purpose of separating serum from a whole blood sample; (b) depicts the inlet through which samples will be introduced, and the beginning of the first module; (c) depicts a junction where the straight path leads to a collection deposit for heavier and irrelevant parts of the sample while the two bifurcations flow the serum portion of the sample; (d) depicts a junction where the serum sample from the two bifurcations meet and follow to a straight channel, and the end of the first module and the beginning of the second module; (e) depicts two sets of acoustic transducers on either side of a straight channel used for the purpose of releasing microRNAs from proteins and complexes in the serum channel; (f) depicts a filter that is used to separate relevant target molecules such as microRNAs from larger molecules such as proteins in the serum sample, and the end of the second module and the beginning of the third module; (g) depicts an intricate microfluidics channel laid over a microarray chip used for the purpose of hybridizing target microRNAs to capture probes; (h) depicts the slot through which chips are inserted and removed. before and after hybridization, respectively, and the end of the third and final module.

FIG. 3 depicts, in accordance with an embodiment described herein, an illustration of the wireframe corner-view of a microfluidics device. (a) depicts an acoustic transducer used for the purpose of separating serum from a whole blood sample; (b) depicts the inlet through which samples will be introduced, and the beginning of the first module; (c) depicts a junction where the straight path leads to a collection deposit for heavier and irrelevant parts of the sample while the two bifurcations flow the serum portion of the sample; (d) depicts a junction where the serum sample from the two bifurcations meet and follow to a straight channel, and the end of the first module and the beginning of the second module; (e) depicts two sets of acoustic transducers on either side of a straight channel used for the purpose of releasing microRNAs from proteins and complexes in the serum channel; (f) depicts a filter that is used to separate relevant target molecules such as microRNAs from larger molecules such as proteins in the serum sample, and the end of the second module and the beginning of the third module; (g) depicts an intricate microfluidics channel laid over a microarray chip used for the purpose of hybridizing target microRNAs to capture probes; (h) depicts the slot through which chips are inserted and removed before and after hybridization, respectively, and the end of the third and final module.

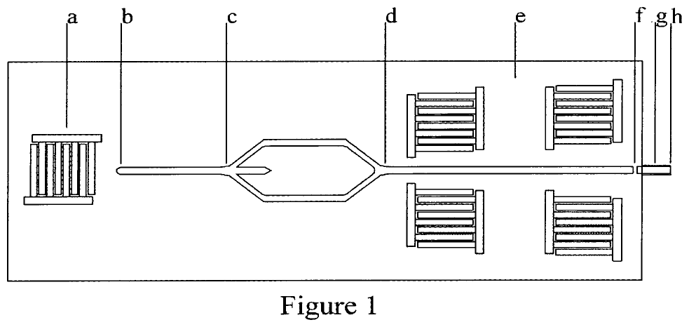

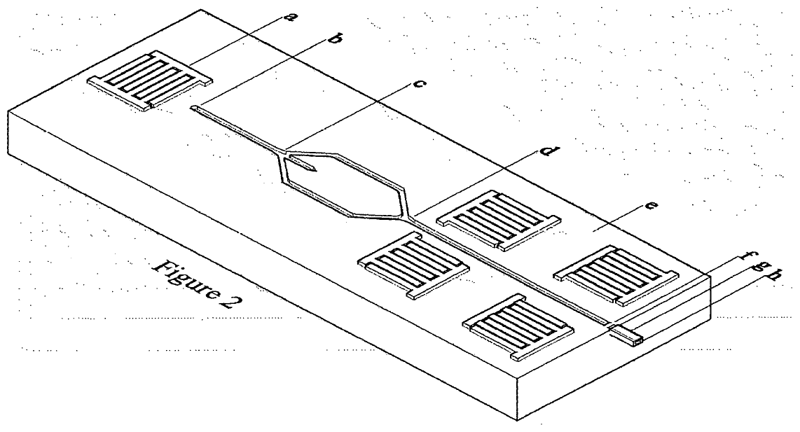

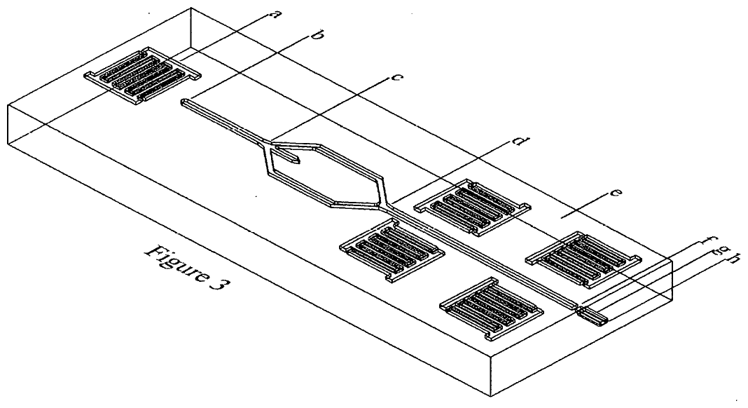

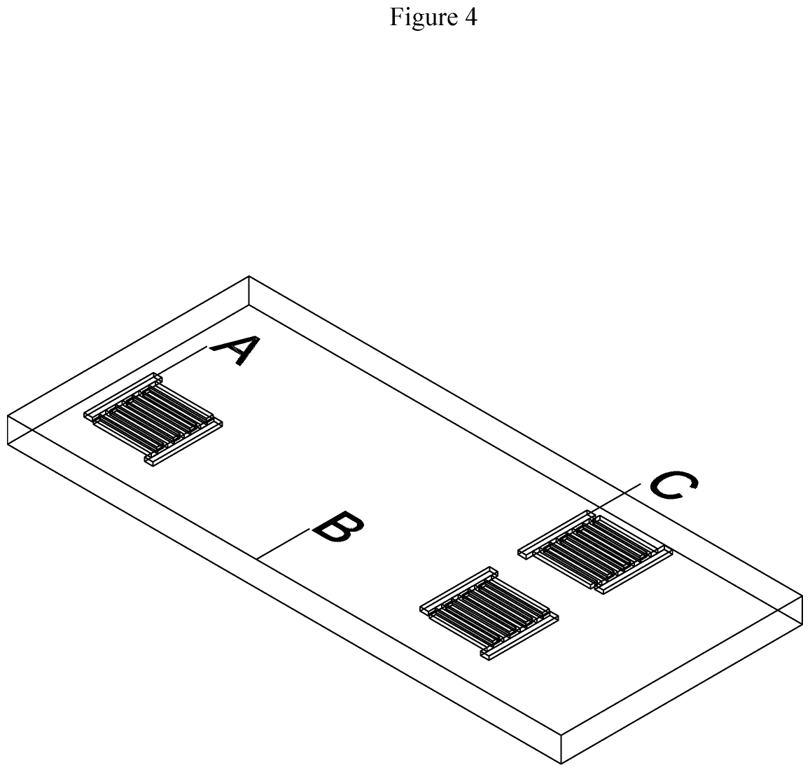

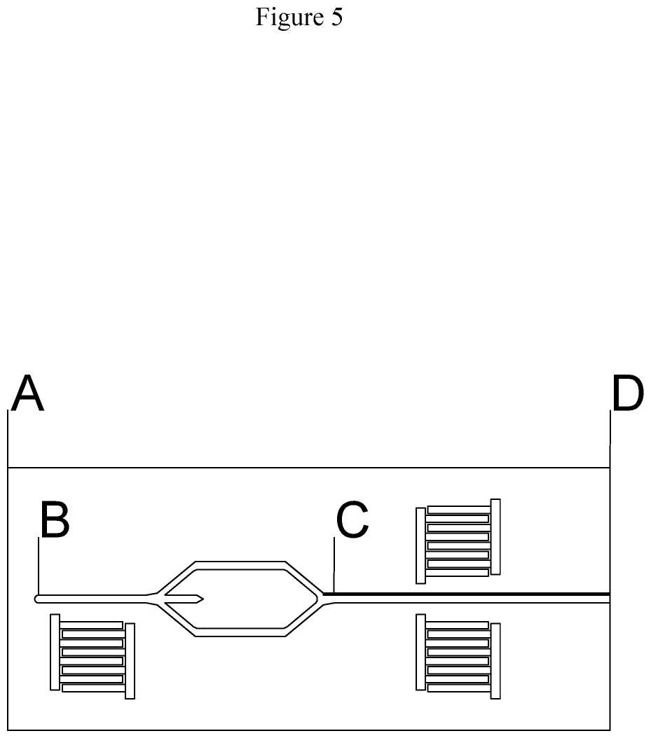

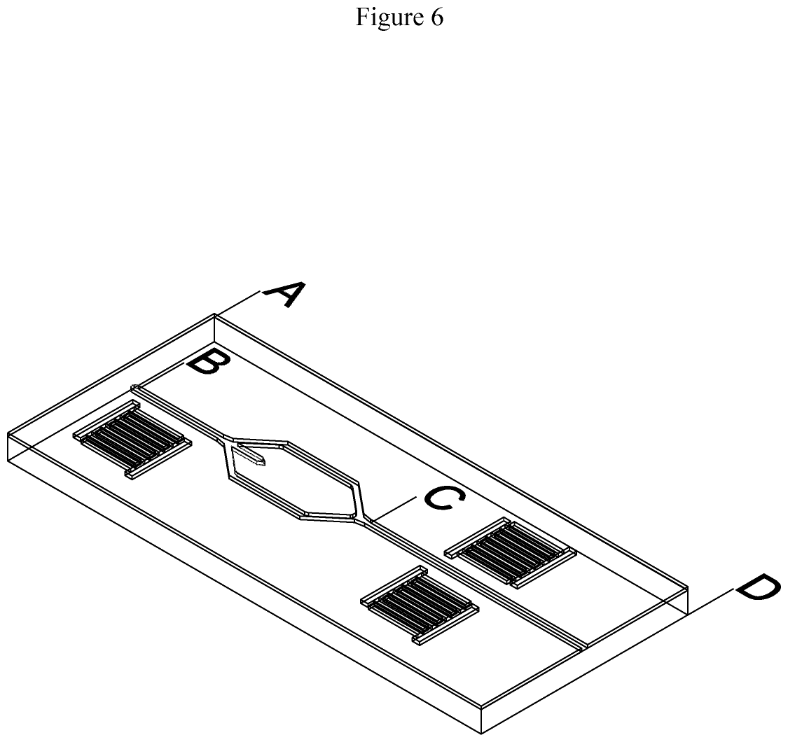

### DETAILED DESCRIPTION OF THE INVENTION

I. Microfluidics Device

As disclosed herein, the inventor created microfluidic devices comprising single or multiple stages with one inlet to introduce a sample and one output to release a subset of analytes. The stages comprise of a channel and an integrated system where a combination of a number of techniques and forces can be applied. The target subset of analytes to be released comes from a predetermined spot on a spectrum of biomolecules based on size and type. Numerous setups are possible, but the key value in the device is the underlying simplicity and flexibility of taking a large sample and outputting a smaller subset of targets.

All biomolecules, molecular coatings, chemicals, materials, processes, and systems mentioned in the following are embodiments of the invention. Any of the thereof can be altered or changed provided the embodiments fit the claims.

II. Microfluidics Device Embodiment—General

One embodiment of this invention is disclosed herein, where a multiple module microfluidics device is used for the purpose of separating and extracting total RNA from a whole blood sample in order to profile microRNA amounts in an automated and mechanical fashion. The microfluidics device is patterned with a design that corresponds to each of the three modules—blood to serum separation, total RNA (all forms of RNA, i.e. microRNA) release from serum, and microRNA hybridization. The first two modules are integrated with different acoustics-based systems for the purpose of separating and releasing, respectively. The third module is fitted with a filtration system to help in extracting the total RNA and leaving larger, irrelevant molecules behind.

III. Microfluidics Device Embodiment—Blood to Serum Acoustics

The first goal of the device is to separate a whole blood sample to serum. There are various methods of accomplishing this such as centrifugation, sedimentation, and chemical processes, but in this embodiment, acoustics is used.

IV. Microfluidics Device Embodiment—Blood to Serum Module Layout

The first module has 3 channels with the main straight channel bifurcating into two other channels at a 3-way junction. An acoustic transducer is placed at the head of the straight channel and aligned to the direction of flow such that heavier molecules (i.e. fibrinogen, clotting factors, i.e.) are pushed forward to one of the 3 channels that lies directly ahead of the straight channel and junction. Such molecules are considered waste and collected in a collection deposit. The residual and relevant molecules of smaller mass which amount to the serum portion of the sample are pushed towards the sides and follow down the bifurcation channels. They converge at a junction further along which merges into a straight path leading the second module.

V. Microfluidics Device Embodiment—Collection Deposit Removal

Heavier molecules which were collected in a waste collection deposit are irrelevant to the goals of this embodiment and as such will be removed or discarded. Such can be done using basic techniques.

VI. Microfluidics Device Embodiment—MicroRNA Separation in Serum Acoustics

The next and main goal of the device is to release microRNAs from various proteins and complexes (i.e. RISCs). There are various methods of accomplishing this with the majority using chemical-based techniques, but in this embodiment, mechanical separation and more specifically, acoustics is used.

VII. Microfluidics Device Embodiment—Serum MicroRNA Release Module Layout

The next module has a straight channel throughout but is lined with 2 pairs of acoustic transducers on either side of the channel, equidistant from each other. The transducers are perpendicular to the direction of flow. Upon passing the acoustic transducers through the length of the channel, molecules bound to proteins and complexes such as microRNAs, are released and become free-floating within the sample. Following the acoustic-based release module of the device, the straight channel leads into a filter which marks the beginning of the third module.

VIII. Microfluidics Device Embodiment—MicroRNA Hybridization

This final module has a filtration system based on size differentiation which allows smaller molecules of a defined size, aligned with our target molecule to pass through, while preventing larger molecules in the sample which are irrelevant to this embodiment from passing through. As such, a set of small molecules called total RNA (a number of RNA types including microRNAs) passes through to an intricate microfluidics pattern which I laid over a microarray chip that has capture probes for specific target microRNAs. As the sample flows through the microfluidic channels and over the chip, target microRNAs hybridize to their respective capture probes. For every run, a used chip is removed and a new one is used. This completes the third module and accomplishes the goal of the microfluidics device in this embodiment.

IX. Microfluidics Device Embodiment—MicroRNA Characterization

The modules of the physical microfluidics device are complete, but as used in this embodiment for microRNA profiling, there are further steps. The microarray chip containing the hybridized microRNAs is washed and placed into a characterizer which reads the expression value for each microRNA using an electrical system. The characterizer is similar to the size of the chip or a US quarter.

X. Microfluidics Device Embodiment—Diagnostics

In this embodiment, the data from the characterizer is recorded and put through specific algorithms for the purpose of learning patterns and reading specific signatures and signs that can be used for early stage diagnostics for an assortment of diseases.

That which is claimed:

1. A microfluidic device comprising: single or multiple stages with one inlet to introduce a sample and one output to release a subset of analytes.

2. The microfluidic device in claim 1, wherein the stages comprise a channel and an integrated system.

3. The microfluidic device's stages in claim 2, wherein the integrated system comprises acoustic, magnetic, thermal, chemical, electric, pressure, and/or mechanical systems.

4. The microfluidic device in claim 1, wherein the stages are modules designated for different target subsets comprising serum, plasma, exosomes, polynucleotides, and/or polypeptides.

6. The microfluidic device in claim 1, wherein the stages are connected and continuous.

7. The microfluidic device in claim 1, wherein the analytes comprise blood fractions, polynucleotides, and/or polypeptides.

8. The microfluidic device in claim 1, wherein the sample comprises blood, plasma, serum, urine, saliva, and tears.
